(19) United States
(12) Patent Application Publication
(10) Pub. No.: US 2026/0083559 A1
(43) Pub. Date: Mar. 26, 2026

(54) Semi-Automated Crimper for Balloon Expandable Heart Valves

(71) Applicant: St. Jude Medical, Cardiology Division, Inc., St. Paul, MN (US)

(72) Inventors: William H. Peckels, Robbinsdale, MN (US); Craig Petersen, Plymouth, MN (US)

(73) Assignee: St. Jude Medical, Cardiology Division, Inc., St. Paul, MN (US)

(21) Appl. No.: 19/276,820

(22) Filed: Jul. 22, 2025

Related U.S. Application Data
(60) Provisional application No. 63/697,848, filed on Sep. 23, 2024.

(51) Int. Cl. A61F 2/24; A61F 2/70; A61F 2/95

(52) U.S. Cl. CPC A61F 2/2433; A61F 2/70; A61F 2/9524; A61F 2002/701

(57) ABSTRACT

A system for crimping and expanding a medical device may include a delivery system with a handle, a catheter, and an inflatable balloon. A syringe may receive inflation media and may be configured to be received on a balloon inflation system and to be coupled to the delivery system. A crimper may have a collapsible iris assembly configured to receive the medical device therein. The crimper may be configured to mechanically couple to the balloon inflation system. The balloon inflation system may actuate the crimper to crimp the medical device onto the balloon when the medical device is received within the iris assembly. The balloon inflation system may be configured to actuate the syringe to advance the inflation media from the syringe to the balloon to expand the balloon and to expand the medical device that is crimped onto the balloon.

### CROSS-REFERENCES TO RELATED APPLICATIONS

This application claims the benefit of priority to the filing date of U.S. Provisional Patent Application No. 63/697,848, filed Sep. 23, 2024, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

### SUMMARY OF THE DISCLOSURE

According to one aspect of the disclosure, a system for crimping and expanding a medical device includes a delivery system having a handle, a catheter extending from the handle, and an inflatable balloon near a distal end of the catheter. The system may also include a balloon inflation system and a syringe configured to receive inflation media therein, the syringe configured to be received on the balloon inflation system and to be coupled to the delivery system. The system may further include a crimper. The crimper may have an iris assembly configured to receive the medical device therein. The iris assembly may be configured to transition from a first larger diameter to a second smaller diameter, and the crimper may be configured to mechanically couple to the balloon inflation system. When the crimper is mechanically coupled to the balloon inflation system, the balloon inflation system may be configured to actuate the crimper to transition the iris assembly from the first larger diameter to the second smaller diameter to crimp the medical device onto the balloon when the medical device is received within the iris assembly. When the syringe (i) has inflation media received therein, (ii) is received on the balloon inflation system, and (iii) is coupled to the delivery system, the balloon inflation system may be configured to actuate the syringe to advance the inflation media from the syringe to the balloon to expand the balloon and to expand the medical device that is crimped onto the balloon. The balloon inflation system may include a moving member and a cradle, the moving member being translatable toward or away from the cradle. The syringe may have a barrel configured to be received within the cradle, and a plunger handle configured to couple to the moving member. The crimper may include a spur gear coupled to a cam track so that rotation of the spur gear causes rotation of the cam track, and rotation of the cam track may transition the iris assembly from the first larger diameter to the second smaller diameter. The crimper may include a rack gear, the rack gear being translatable into or out of a base of the crimper, translation of the rack gear causing rotation of the spur gear. The rack gear may include a rack gear handle, the rack gear handle configured to be coupled to the moving member of the balloon inflation system. The crimper may include a distal projection, the distal projection configured to be received within the cradle of the balloon inflation system. The balloon inflation system may include a motor. The crimper may exclude a motor. The crimper may be configured to be electrically connected to the balloon inflation system. The crimper may include at least one sensor. The sensor may be a position sensor and/or a force sensor. The system may include the medical device. The medical device may be a balloon-expandable prosthetic heart valve.

According to another aspect of the disclosure, a method may include positioning a medical device on an inflatable balloon that is coupled to a distal end of a catheter of a delivery system while the medical device is in an expanded condition. While the medical device is in the expanded condition on the balloon, the balloon and the medical device may be positioned within an iris assembly of a crimper while the iris assembly has a first large diameter. The crimper may be mechanically coupled to a balloon inflation system, either before or after positioning the balloon and the medical device within the iris assembly of the crimper. The balloon inflation system may be actuated a first time, while the crimper is mechanically coupled to the balloon inflation system, to transition the iris assembly from the first large diameter to a second smaller diameter to crimp the medial device into a collapsed condition on the balloon. The method may also include mechanically uncoupling the crimper from the balloon inflation system after the medical device is crimped on the balloon, and then mechanically coupling a syringe to the balloon inflation system, the syringe having inflation media therein. The medical device may be a balloon-expandable prosthetic heart valve, and the method may further include advancing the crimped prosthetic heart valve into a patient until the crimped prosthetic heart valve is positioned within a native valve annulus of a patient. The method may also include actuating the balloon inflation system a second time, while the crimped prosthetic heart valve is positioned within the native valve annulus of a patient, to advance inflation media from the syringe, through the delivery system, and into the balloon to inflate the balloon and to expand the prosthetic heart valve into the native valve annulus of the patient. Actuating the balloon inflation system the first time may include activating a motor within the balloon inflation system to translate a moving member of the balloon inflation system, whereby the moving member advances a rack gear of the crimper to rotate a spur gear of the crimper, rotation of the spur gear rotating a cam track such that rotation of the cam track transitions the iris assembly from the first large diameter to the second smaller diameter.

According to still a further aspect of the disclosure, a system for crimping and expanding a medical device may include a delivery system having a handle, a catheter extending from the handle, and an inflatable balloon near a distal end of the catheter. The system may include a balloon inflation system, and a syringe configured to receive inflation media therein. The syringe may be configured to be received on the balloon inflation system and to be coupled to the delivery system. The system may also include a crimp. The crimper may have an iris assembly configured to receive the medical device therein. The iris assembly may be configured to transition from a first larger diameter to a second smaller diameter. The crimper may be configured to electrically couple to the balloon inflation system. When the crimper is electrically coupled to the balloon inflation system, the balloon inflation system may be configured to actuate a motor within the crimper to transition the iris assembly from the first larger diameter to the second smaller diameter to crimp the medical device onto the balloon when the medical device is received within the iris assembly. When the syringe (i) has inflation media received therein, (ii) is received on the balloon inflation system, and (iii) is coupled to the delivery system, the balloon inflation system may be configured to actuate the syringe to advance the inflation media from the syringe to the balloon to expand the balloon and to expand the medical device that is crimped onto the balloon. The crimper may include a spur gear coupled to a cam track so that rotation of the spur gear causes rotation of the cam track, and rotation of the cam track transitions the iris assembly from the first larger diameter to the second smaller diameter. The crimper may include a pinion gear, the pinion gear having teeth that intermesh with teeth of the spur gear such that rotation of the pinion gear causes rotation of the spur gear. The balloon inflation system may be configured to actuate the motor within the crimper by transmitting power to the motor to cause the motor to rotate the pinion gear. The balloon inflation system may be configured to actuate the crimper without requiring any mechanical connection between the balloon inflation system and the crimper.

According to a further aspect of the disclosure, a method includes positioning a medical device on an inflatable balloon that is coupled to a distal end of a catheter of a delivery system while the medical device is in an expanded condition. The method may include, while the medical device is in the expanded condition on the balloon, positioning the balloon and the medical device within an iris assembly of a crimper while the iris assembly has a first large diameter. The crimper may be electrically coupled to a balloon inflation system, either before or after positioning the balloon and the medical device within the iris assembly of the crimper. The balloon inflation system may be actuated a first time, while the crimper is electrically coupled to the balloon inflation system, to transition the iris assembly from the first large diameter to a second smaller diameter to crimp the medial device into a collapsed condition on the balloon. A syringe may be mechanically coupled to the balloon inflation system, the syringe having inflation media therein. The medical device may be a balloon-expandable prosthetic heart valve, and the method may further include advancing the crimped prosthetic heart valve into a patient until the crimped prosthetic heart valve is positioned within a native valve annulus of a patient. The balloon inflation system may be actuated a second time, while the crimped prosthetic heart valve is positioned within the native valve annulus of a patient, to advance inflation media from the syringe, through the delivery system, and into the balloon to inflate the balloon and to expand the prosthetic heart valve into the native valve annulus of the patient. Actuating the balloon inflation system the first time may include transmitting power from the balloon inflation system to a motor within the crimper to rotate a pinion gear within the crimper, whereby rotating the pinion gear rotates a spur gear and a cam track coupled thereto, such that rotation of the cam track transitions the iris assembly from the first large diameter to the second smaller diameter.

According to still another aspect of the disclosure, a system for use with a medical device may include a crimper, a first pinion gear, and a motor within the crimper operably coupled to the first pinion gear so that activation of the motor causes rotation of the first pinion gear in a first rotational direction. The crimper may include a second gear having gear teeth operably engaged with gear teeth of the first pinion gear so that rotation of the first pinion gear in the first rotational direction causes rotation of the second gear in a second rotational direction opposite the first rotational direction. The crimper may include a plurality of contact members, each contact member having a contact arm so that the plurality of contact arms collectively form an iris opening having a central longitudinal axis, whereby rotation of the second gear moves the plurality of contact arms toward or away from the central longitudinal axis to change a size of the iris opening. The system may include a power source within the crimper. The power source may be a rechargeable battery. The system may include a delivery system having a handle, a catheter extending from the handle, and an inflatable balloon near a distal end of the catheter, the balloon configured to receive the medical device thereon. The system may also include a balloon inflation system and a computer tablet device. The balloon inflation system may include a rechargeable battery, and the computer tablet device may include a rechargeable battery. The system may include a charging dock configured to receive thereon each of the crimper, the balloon inflation system, and the computer tablet device, the charging dock configured to charge the respective rechargeable battery of each of the crimper, the balloon inflation system, and the computer tablet device. The crimper may be configured to be in wireless communication with the computer tablet device such that the computer tablet device is configured to activate the motor of the crimper. The balloon inflation system may be configured to receive a syringe with inflation media while the syringe is coupled to the delivery system such that activation of the balloon inflation system forces inflation media from the syringe, through the delivery system, and into the balloon to expand the balloon and the medical device received thereon. The balloon inflation system may include a motor, such that actuation of the motor activates the balloon inflation system to force inflation media from the syringe. The balloon inflation system may be configured to be in wireless communication with the computer tablet device such that the computer tablet device is configured to activate the motor of the balloon inflation system.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the handle of the delivery system shown in FIG. 4.

FIG. 6 is an enlarged view of a distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the inflation system of FIG. 8.

FIG. 10 is a perspective view of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12A is a side view of a manual mechanical crimper according to an aspect of the disclosure.

FIG. 12B is a perspective view of the manual crimper of FIG. 12A with certain components being shown in phantom.

FIG. 13A is a side view of the crimper of FIG. 12A with certain components shown in phantom.

FIG. 13B is a side view of the crimper of FIG. 13A, with certain components removed from the view to better see interior components.

FIG. 13C provides the same view as FIG. 13B, with a first crimping guide shown in addition to the components of FIG. 13B, the first crimping guide shown in phantom.

FIG. 13D provides the same view as FIG. 13B, with a second crimping guide shown in addition to the components of FIG. 13B, the second crimping guide shown in phantom.

FIG. 14A is a side view of an automated or semi-automated crimper in accordance with an aspect of the disclosure, the crimper being shown in a generally open state.

FIG. 14B is side view of the crimper of FIG. 14A, the crimper being shown in a generally closed state.

FIGS. 14C-14D are side views of the crimper of FIGS. 14A-14B, respectively, coupled to an inflation system similar to the balloon inflation system shown in FIGS. 8-10.

FIG. 14E is a schematic view of the connection between the crimper and the inflation system of FIGS. 14C-14D.

FIG. 15 is a schematic view of a balloon inflation system similar to the balloon inflation system shown in FIGS. 8-10 electrically coupled to a crimper according to another aspect of the disclosure.

FIG. 16A is a schematic view of a crimper according to another aspect of the disclosure.

FIG. 16B is a schematic view of the crimper of FIG. 16A, a balloon inflation system similar to those shown in FIGS. 8-10, and a tablet device all coupled to a docking station.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

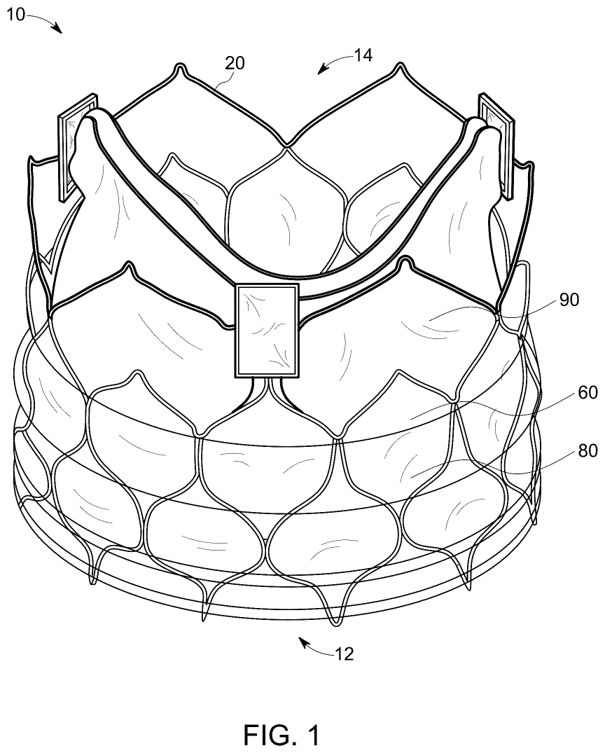

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

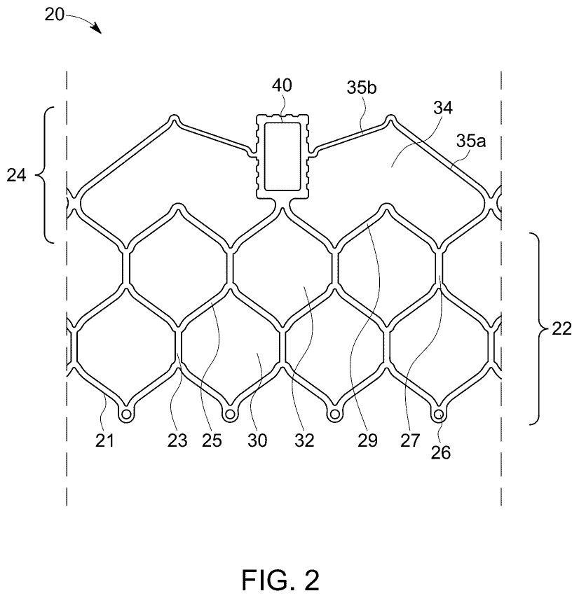

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g. fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Patent Application Publication No. 2025/0073023, the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g. struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

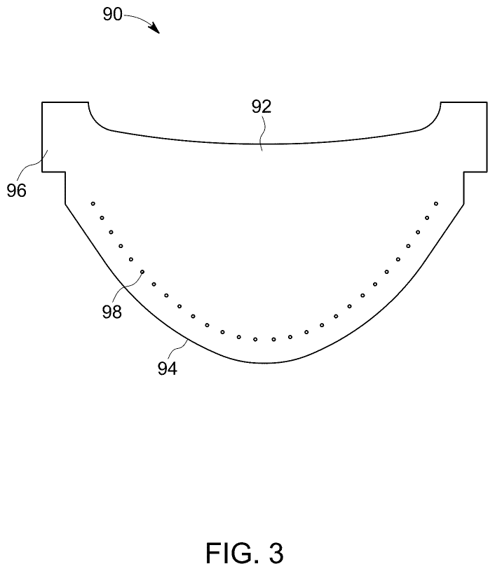

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g. via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Laflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

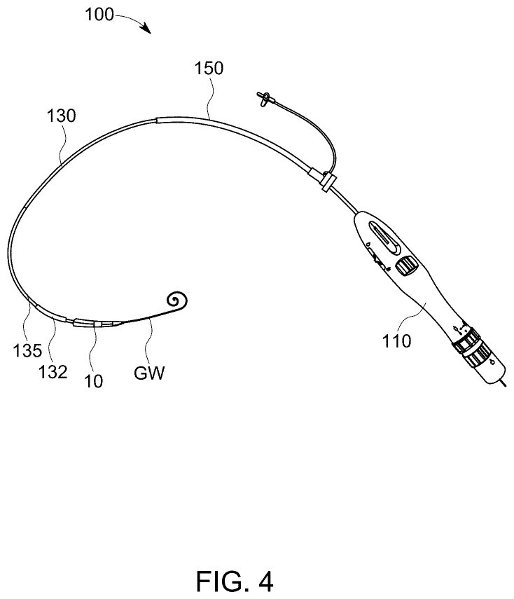

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

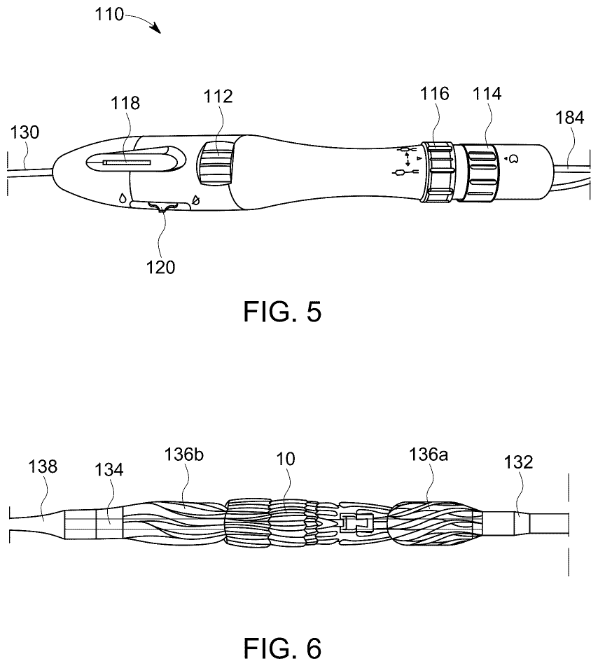

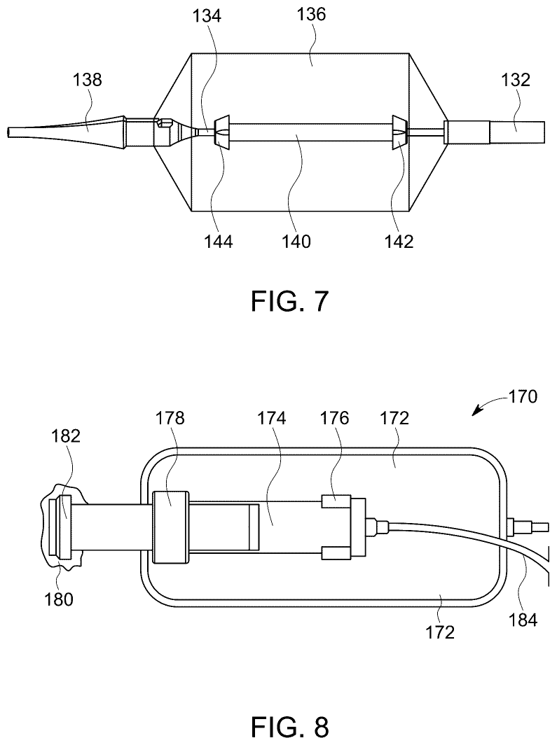

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135. In some examples, the handle may include a window 118 that allows viewing of an indicator that corresponds to the amount of catheter deflection. For example, a carrier to which the indicator is attached may be attached to the steering wire. In some examples, when there is minimum (or zero) tension on the steering wire, the indicator is at the far distal position within window 118, but as deflection is actuated, for example by drawing a carrier proximally (and tensioning the steering wire as the carrier draws proximally), the indicator will move proximally along window 118, giving the user a readily-apparent indication of the amount of deflection applied to the catheter at any given moment.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads (or another component, such as individual extensions, which may be cylindrical extensions that fit between internal threads of the actuator) of a carriage that is coupled to an inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it may have a small total range of motion, including for example between about 2 mm and about 15 mm of range of motion, including about 7.5 mm range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Patent Application Publication No. 2024/0148501, the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. In some embodiments, the balloon actuator 120 may take the form of a “momentary switch” in which pushing the balloon actuator 120 forward engages inflation, pulling the balloon actuator 120 proximally engages deflation, and releasing the balloon actuator 120 pauses inflation. This particular example of functionality may allow the physician to precisely control the amount of fluid dispensed while reducing the occurrence of over- or under-inflation, for example because the system automatically pauses inflation when the switch is released. The physical form factor of the balloon actuator 120 may be any suitable desired form factor, including for example a rocker switch, a push button, etc. In some embodiments a second balloon actuator or button may be provided, either on the balloon actuator 120 or elsewhere on the handle 110, with the second balloon actuator allowing for a change (e.g. increase or decrease) in the rate of inflation, for example to a pre-programmed faster or slower rate of inflation. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

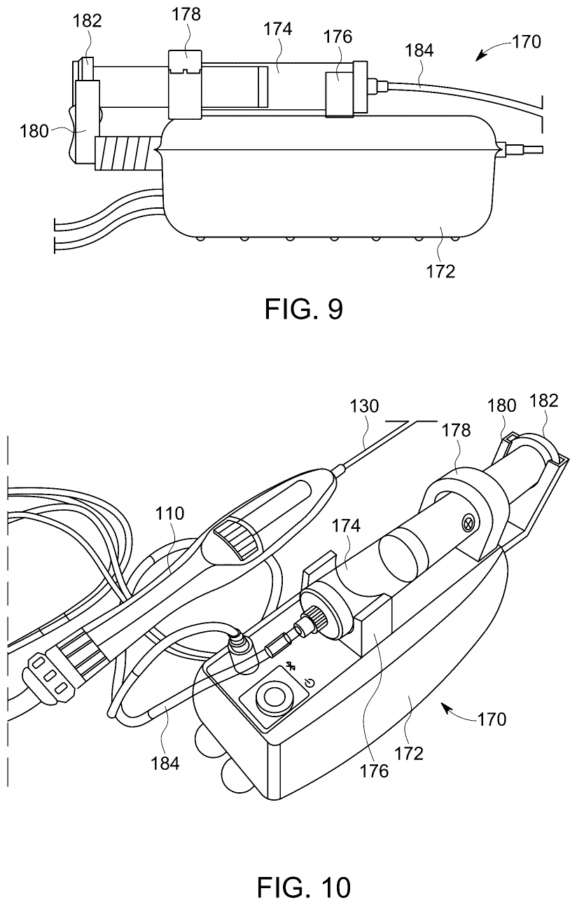

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g. from AC mains or another component with which the cable is coupled) and/or transmission of data, information, control commands, etc. For example, one cable may couple the housing 172 to handle 110 so that controls on the handle 110 (e.g. balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. Patent Application Publication No. 2023/0372097, the disclosure of which is hereby incorporated by reference herein.

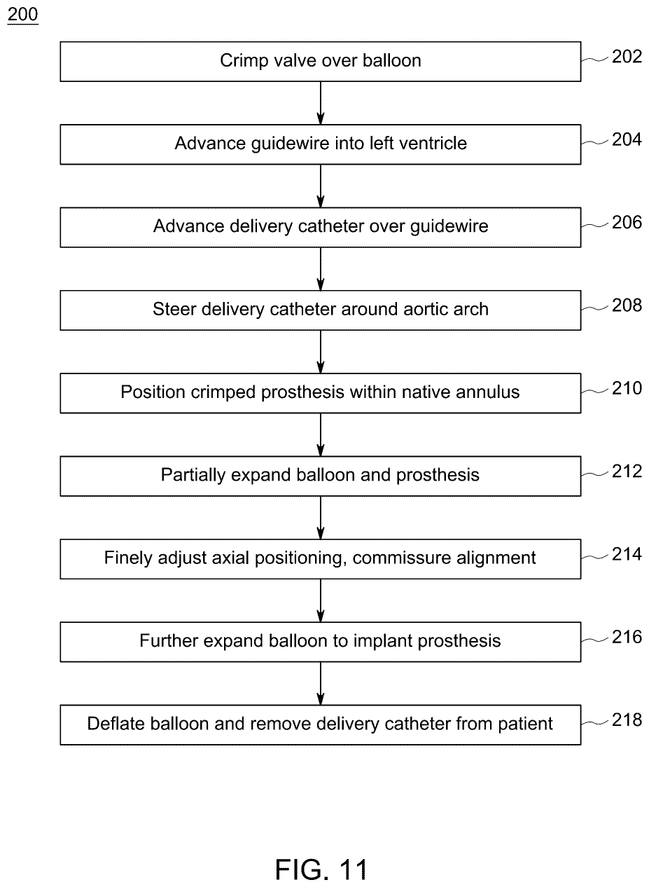

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g. by actuating axial alignment actuator 116) and/or the rotational orientation of the prosthetic heart valve 10 relative to the native aortic valve may be finely adjust (e.g. by actuating commissure alignment actuator 114). When the desired axial alignment is achieved and the desired rotational alignment (e.g. rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

As noted above, balloon expandable prosthetic heart valves, including prosthetic heart valve 10, are typically forcibly collapsed or crimped onto a delivery device just prior to being implanted into a patient, for example onto balloon 136 of delivery system 100, or onto another component of the delivery system. And while this disclosure focuses on crimping devices that are useful for forcibly collapsing balloon expandable prosthetic heart valves, it should be understood that the crimping devices described herein may be suitable for crimping or forcibly collapsing other types of devices that incorporate plastically expandable frames, including stents and related stent-like devices.

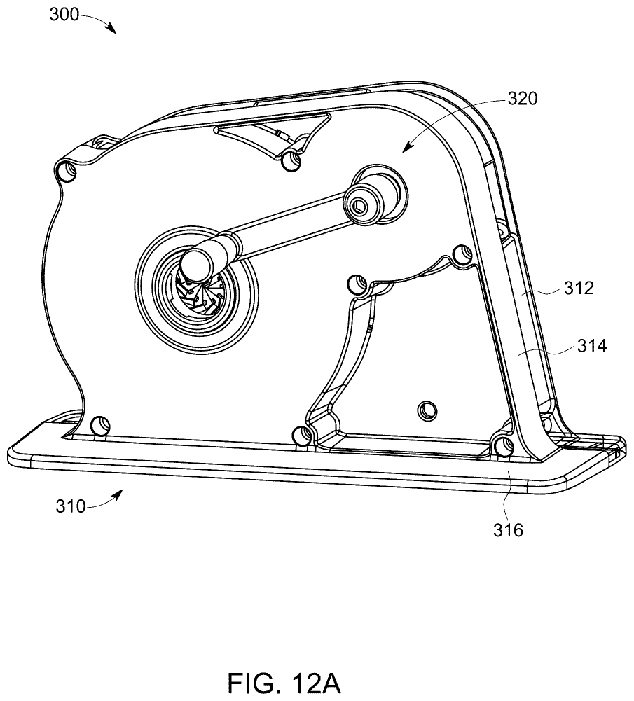

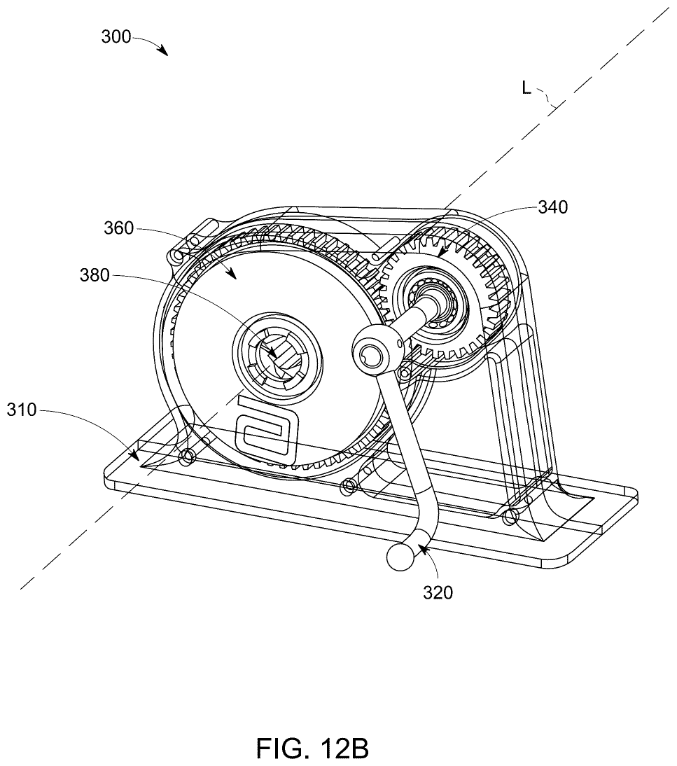

One example of a manual crimping device 300 (also referred to as a “crimper”) is shown in FIG. 12A. FIG. 12B shows the crimping device 300 with a casement or housing assembly 310 thereof in partial phantom. As shown in FIGS. 12A-12B, the crimping device 300 may generally include a housing assembly 310, an actuator or handle assembly 320, a pinion or first gear assembly 340, a large or second gear assembly 360, and an iris assembly 380. It should be understood that, although the word “assembly” is used above, an “assembly” in some embodiments may include either a single component or multiple components that are structurally or functionally related. On a general level, the crimper 300 may be used by placing a device (such as a prosthetic heart valve, which may be positioned on a balloon or another component of a delivery device, or such as a stent) into the iris assembly 380 while the device is in a relatively expanded condition and while the iris assembly 380 is in a relatively opened condition. Then, the user may manually rotate the handle assembly 320 to cause the pinion assembly 340 to rotate, which in turn causes the large gear assembly 360 to rotate, which in turn forces the iris assembly 380 to transition to a relatively closed state, forcibly collapsing the device therein into a relatively collapsed condition. It should be understood that crimper 300 is merely one example of a manual crimper which may be modified for use in a semi-automated fashion as described in greater detail below. Other examples of manual crimpers may be modified for use in a semi-automated fashion as described in greater detail below, but manual crimper 300 is first described in more detail to provide context for the general working of one example of a manual crimper.

Referring generally to FIGS. 12A-12B, the housing assembly 310 may include a rear shell 312 and a front shell 314 which may, when assembled, encapsulate or cover most of the components of the crimper 300, with certain exceptions, including for example portions of the handle assembly 320 and the iris assembly 380. The rear shell 312 and front shell 314, when assembled, may form a relatively small compartment to house the pinion assembly 340 and a relatively large compartment to house the large gear assembly 360, with the two compartments connected to each other to allow for contact between the pinion assembly 340 and the large gear assembly 360. The housing assembly 310 may also include a base 316, which may include a generally flat inferior surface to allow for the crimper 300 to stably sit on a table during use.

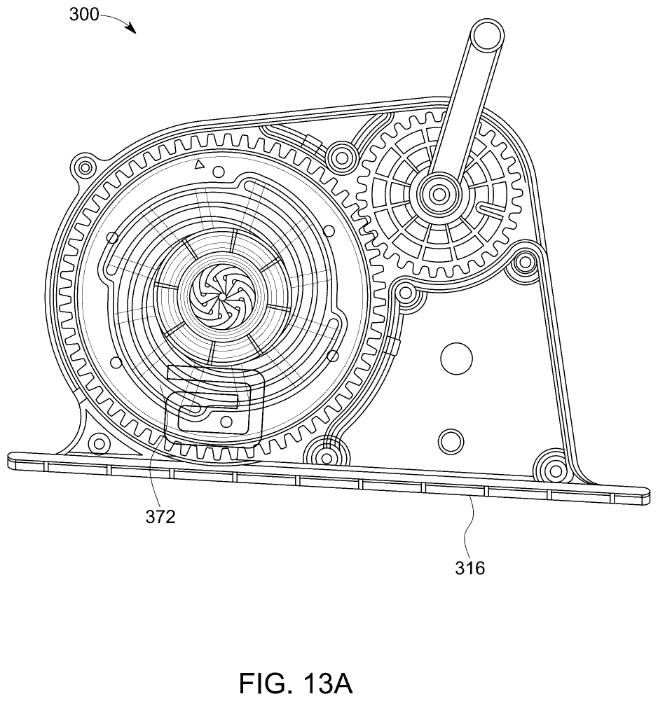

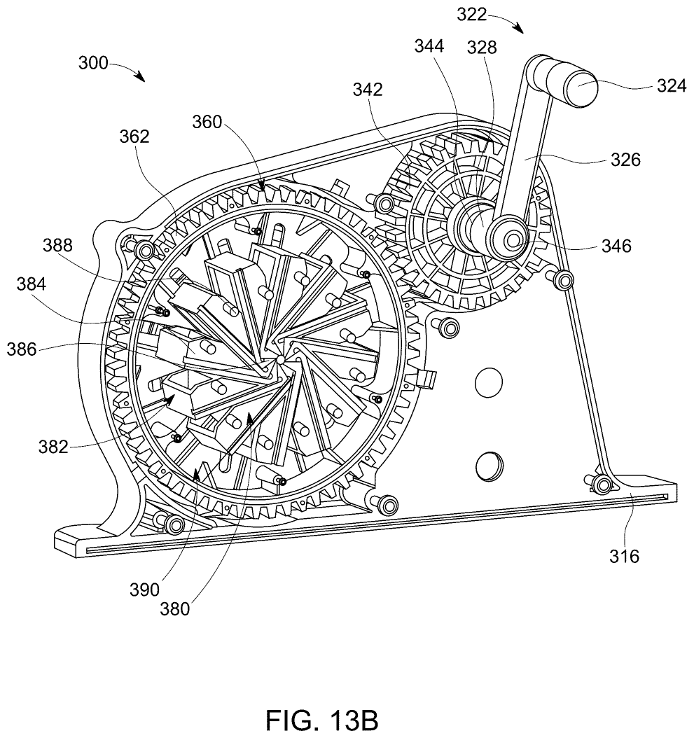

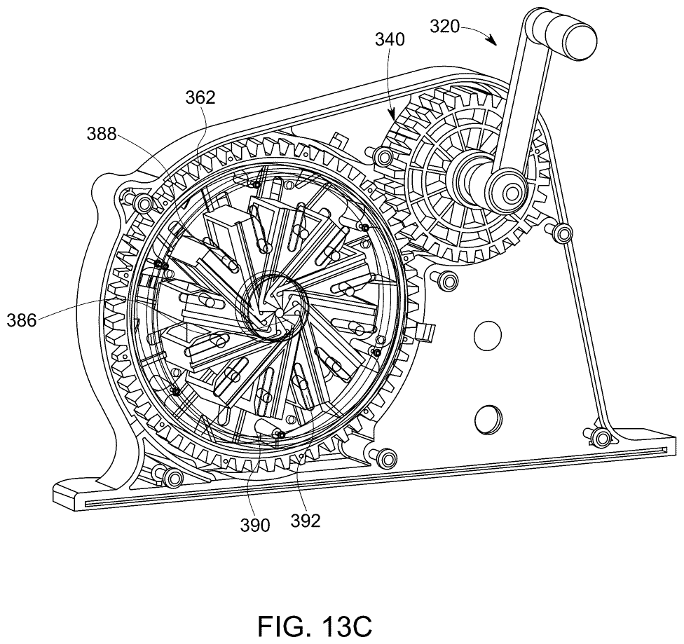

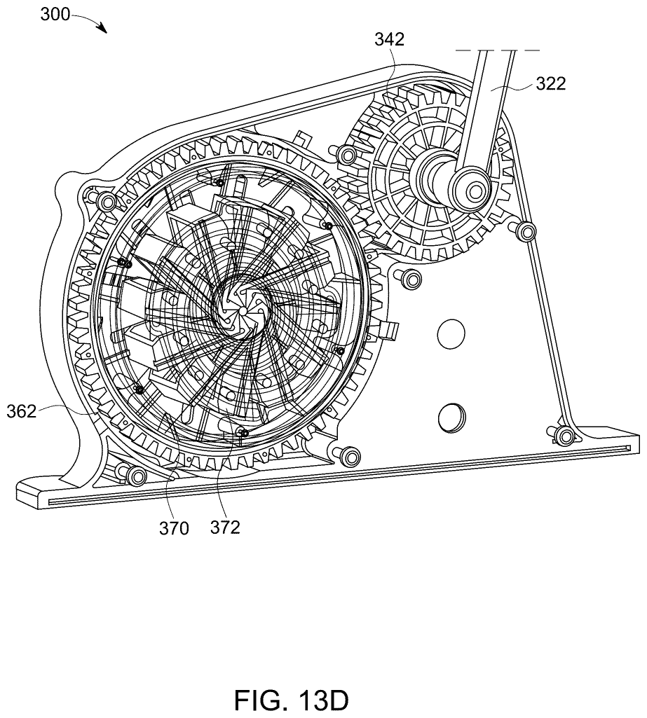

FIG. 13A shows a side view of crimper 300 with the front shell 314 shown in phantom to help illustrate certain internal components of the crimper 300. FIG. 13B shows a similar side view as FIG. 13A, but in FIG. 13B, the front casing 314 has been removed, and the base 316 has been sectioned. As shown in FIG. 13B, the pinion gear assembly 340 may include a pinion 342 which may take the form of a spur gear. The pinion 342 may have a plurality of pinion teeth extending radially outwardly from an outer circumference thereof, and may define an interior opening along a central axis of the pinion 342. A pinion drive shaft 344 may pass into the interior opening, and the pinion drive shaft 344 and interior opening of the pinion 342 may be geometrically (or otherwise) keyed so that rotation of the pinion drive shaft 344 causes rotation of the pinion 342. In other words, the pinion drive shaft 344 is rotatably fixed to the pinion 342, while the pinion 342 is suspended within the housing assembly 310 so that, when the pinion 342 rotates, it does not contact any surfaces of the housing assembly 310 that would hinder rotation of the pinion 342.

Although not shown in FIG. 13B (see instead, e.g., FIG. 12A), the pinion drive shaft 344 may extend outwardly form the front casing 314 when the housing assembly 310 is fully assembled. The handle assembly 320 may include a handle 322, which may include a first handle end 324 configured to be gripped by a user to actuate pinion gear 342. The handle 322 may include a main handle body 326 extending from the first handle end 324 to a second handle end 328. The second handle end 328 may be hollow so that the exposed portion of the pinion drive shaft 344 is received within the second handle end 328. In some examples, a fastener 346 may be provide to fasten the handle 322 to the pinion drive shaft 344, for example due to the fastener 346 having a head with a larger dimension than the interior dimension of the second handle end 328. The second handle end 328 is rotationally fixed to the pinion drive shaft 344, for example via geometric keying or any other suitable fashion, such that rotation of the handle 322 transmits torque to the pinion drive shaft 344, which in turn transmits torque to the pinion gear 342, such that one rotation of the handle 322 creates one rotation of the pinion 342. Although one particular example of handle assembly 320 is described herein, it should be understood that other specific configurations of handles may be implemented in order to allow transmission of torque between handle 322 and pinion 342. In at least some examples, the center axis of the pinion 342 is positioned above (i.e. upward when the base 316 of the crimper 300 sits on a table) the central axis of the iris assembly 380.

Still referring to FIG. 13B, the large gear assembly 360 may include a large gear 362 which may take the form of a spur gear. The large gear 362 may have a plurality of gear teeth extending radially outwardly from an outer circumference thereof, and may have a largely hollow interior that houses other components of the large gear assembly 360 and/or the iris assembly 380. Not shown in FIG. 13B, although shown in other figures and described in greater detail below, the large gear assembly 360 may include a cam track 370 which is rotationally fixed to the large gear 362. The cam track 370 may be engaged with components of the iris assembly 380 (described in greater detail below) which are fixed to the housing assembly 310, such that the large gear assembly 360 is effectively suspended within the housing assembly 310, allowing the large gear 362 to rotate without being hindered by contact with components of the housing assembly 310.

Still referring to FIG. 13B, the teeth of pinion 342 intermesh with the teeth of large gear 362 such that rotation of the handle 322 in one rotational direction causes rotation of the large gear 362 in the opposite rotational direction. In some examples, by providing two gears, a mechanical advantage may be obtained. For example, the size of the pinion gear 342 and the size of the large gear 362 may be selected to obtain a desired gear ratio. In one example, the gear ratio may be 2:1. In another example, the gear ratio may be 3:1. For example, the outer diameter of the pinion 342 may be about half of the outer diameter of the large gear 362, or about one-third the diameter of the large gear 362. This mechanical advantage may make it easier for the user to manually rotate the handle 322 to cause crimping of the device placed within the iris assembly 380. For example, with a 2:1 gear ratio, each turn of the large gear 362 will correspond to about two turns of the pinion gear 342, or with a 3:1 gear ration, each turn of the large gear 362 will correspond to about three turns of the pinion gear 342. Although 2:1 and 3:1 are provided as examples of possible gear ratios, it should be understood that sizes of the pinion gear 342 and large gear 362 may be selected to obtain any useful mechanical advantage (e.g. in which more than one revolution of the pinion gear 342 is required to achieve a full revolution of the large gear 362). Thus, although mechanical advantage is desirable, using a gear ratio of greater than 3:1 or greater than 5:1 or greater than 15:1 may be undesirable. Thus, in some examples, the mechanical advantage should be no more than about 15:1.

The way in which rotation of the handle 322 causes crimping of the device within the iris assembly 380 is described below. As noted above, rotating the handle 322 in one rotational direction causes the pinion 342 to rotate in the same rotational direction, and meshing of the pinion 342 and the large gear 362 results in the large gear 362 rotating in the opposite rotational direction as the handle 322.

Referring to FIG. 13B, a portion of the iris assembly 380 is shown with the iris assembly 380 in a closed or substantially closed condition in which an opening defined by a plurality of crimping members 382 (which may also be referred to as contact member or crimping jaws) is at a minimum. In the particular illustrated example, the iris assembly 380 includes a total of twelve contact members 382, although it should be understood that more or fewer may be provided. Each contact member 382 may include a main body 384 which terminates in a contact arm 386 which may extend at an angle relative to the main body 384. The plurality of contact arms 386, in the aggregate, define the opening of the iris assembly 380, with the opening of the iris assembly 380 being relatively small when the contact arms 386 are positioned relatively close to the longitudinal axis L of the iris assembly 380, and being relatively large when the contact arms 386 are positioned relatively far from the longitudinal axis L of the iris assembly 380. It should be understood that it is the exposed surface of the contact arms 386, which are all oriented generally facing the longitudinal axis L of the iris assembly 380, which contact the device within the iris assembly 380 during crimping.

Still referring to FIG. 13B, it should be understood that the contact members 382 of the iris assembly 380 may all be positioned radially within the portion of the large gear 362 that from the gear teeth that mesh with corresponding gear teeth of pinion 342. Each contact member 382 may include at least one protrusion 388 extending therefrom, in a direction generally parallel to the longitudinal axis L of the iris assembly 380. In the illustrated example, each contact member 382 includes two protrusions 388 that are aligned with each other along the same axis, although in FIG. 13B only one protrusion 388 is visible per contact member 382. Preferably, the protrusions are each generally cylindrical or pin shaped. Each protrusion 388 is preferably fixed to the correspond main body 384 of the contact member 382, for example by being formed integrally with the contact member 382, by being adhered to the contact member 382, by being welded to the contact member 382, or by any other suitable mechanism to achieved a fixed relationship.

Although each of the contact members 382 may be substantially identical to each other, groups of the contact members 382 may be provided with their protrusions 388 at slightly different positions. For example, in the specific embodiment of FIG. 13B, a total of twelve contact members 382 are provided in four groups, represented by the “1,” “2,” or “3” printed on the contact member 382 in FIG. 13B. The contact members 382 labeled “1” may include a protrusion 388 relatively far from the contact arm 386, and the contact members 382 labeled “3” may include a protrusion 388 relatively close to the contact arm 386, with the contact members 382 having a protrusion 388 at an intermediate location compared to the other two groups of contact members 382. As should be understood below with the description of cam track 370, this configuration may allow for all protrusions 388 to be received within different positions on a spiral groove 372 of the cam track 370 while maintaining a generally circular opening defined by the contact arms 386.

FIGS. 13A-B also show a first crimping guide 390, which may be part of the iris assembly 380, positioned behind the plurality of contact members 382. The first crimping guide 390 may be positioned generally radially inward of the outer portions of the large gear 362, and the first crimping guide 390 may be fixed to a portion of the housing assembly 310 (e.g. rear shell 312 and/or front shell 314). In some embodiments, a pair of first crimping guides 390 may be provided, such that one first crimping guide 390 is positioned on each side of the plurality of contact members 382. For example, referring now to FIG. 13C, another first crimping guide 390 is shown positioned in front of the plurality of contact members 382. The first crimping guide(s) 390 may be generally circular or cylindrical, and may each define a center opening that has a center axis that is substantially coaxial with the longitudinal axis L of the iris assembly 380. With this configuration, the interior opening of the iris assembly 380 is accessible through the corresponding central opening in the first crimping guide(s) 390. As shown in FIG. 13C, the first crimping guide(s) 390 may include a plurality of radial slots 392 formed therein. Preferably, the first crimping guide(s) 390 have the same number of radial slots 392 as the total number of contact members 382, which is twelve in this particular embodiment. Each radial slot 392 may have a width that is about equal to or slightly larger than the width or diameter of the protrusion 388, and a length that is greater than the width. Preferably, each slot 392 includes a fully enclosed boundary defined by the first crimping guide 390, with the slot 392 extending through an entire thickness of the first crimping guide 390. The longer dimension of each slot 392 (e.g. the length dimension) may extend generally radially toward and away from the radial center of the first crimping guide 390. With this configuration, as described in greater detail below, the protrusion(s) 388 of each contact member 382 may extend through a corresponding slot 392 of the first crimping guide(s) 390. If a pair of first crimping guides 390 is provided, each contact member 382 may include a pair of oppositely extending protrusions 388 extending through corresponding slots 392 of the first crimping guides 390. Whether one or two first crimping guide(s) 390 are provided, the slots 392 may prevent any side-to-side motion of the contact members 382 via the close fit of each protrusion 388 within the width of the corresponding slot 392. As is described in greater detail below, when the second crimping guide or cam track 370 forces the contact members 382 to move radially inwardly or outwardly toward the longitudinal axis L of the iris assembly 380, the slots 392 may both guide and limit the extent of the movement of the contact members 382, via the interaction between each protrusion 388 and its corresponding slot 392. As noted above, whether one or two first crimping guides 390 are provided, each first crimping guide 390 is fixed to the housing assembly 310 so that each first crimping guide 390 remains stationary relative to the housing assembly 310, including when the handle 322 forces the pinion 342 and large gear 362 to rotate.

Referring now to FIG. 13D, crimper 300 may include a second crimping guide 370, which may also be referred to as a cam track 370. In some embodiments, a pair of cam tracks 370 are provided, one on each side of the plurality of contact members 382. The cam track 370 may be thought of as part of the large gear assembly 360. In some embodiments, the cam track(s) 370 is provided outward of the first crimping guide 390. In other words, if two first crimping guides 390 are provided, they may both be positioned between a pair of cam tracks 370. Generally, the cam track(s) 370 may be substantially circular or cylindrical, with an inner opening that generally aligns with the opening of the iris assembly 380 when the crimper 300 is fully assembled so that the device to be crimped may be placed into the interior of the iris assembly 380 through corresponding openings in the housing assembly 310, the cam track(s) 370, and the first crimping guide(s) 390. As best shown in FIGS. 13A and 13D, each cam track 370 may include a plurality of spiral-shaped grooves 372 formed therein. Each spiral-shaped groove 372 may have a first terminal end positioned relatively close to the outer radial edge of the cam track 370, and may extend in a spiraling fashion around the central opening of the cam track 370 to a second terminal end that is positioned relatively close to the central opening of the cam track 370. In other words, the radial position of each groove 372 gradually moves toward the central opening of the cam track 370 from the first terminal end of the groove 372 toward the second terminal end of the groove 372.

The number of grooves 372 provided in the cam track 370 may relate to the number of contact members 382. For example, as noted above, the illustrated example includes a total of twelve contact members 382, arranged as four groups of three contact members 382 per group. Thus, with this configuration, a total of four spiral-shaped grooves 372 may be provided corresponding to the four groups of three contact members 382. The protrusion(s) 388 of each contact member 382 in a single group may extend into the same groove 372, with the different heights or positions of the protrusion(s) 388 in each contact member 382 of the group allowing for the protrusion(s) 388 to fit within different portions of the spiral-shaped groove 372 while the contact members 382 are otherwise in an identical radial position. As should be understood, this positioning may be repeated for each group of contact members 382 for the remaining spiral-shaped grooves 372. Further, it should be understood that each spiral-shaped groove may have a width that is about equal, or slightly larger than, the width or diameter of the protrusion(s) 388, similar to the width of the radial slot(s) 392. The protrusion(s) 388 may have a length that allows them to first pass through the corresponding radial slot(s) 392, and then into the corresponding spiral-shaped groove 372.

Still referring to FIG. 13D, the cam track(s) 370 may be fixed to the large gear 362. For example, fasteners may pass through an outer peripheral portion of the cam track(s) 370 and into a location at an outer periphery of the large gear 362, such that the large gear 362 and the cam track(s) 370 rotate in unison. In some examples, a single cam track 370 is provided, and in other examples, as noted above, a pair of cam tracks 370 may be positioned on each side of the plurality of contact members 382.

In an exemplary use of crimper 300, the iris assembly 380 may have an initial configuration in which the opening of the iris assembly 380 is maximally open, which may correspond to each of the contact members 382 (and particularly contact arms 386) being positioned their maximum radial distance away from the longitudinal axis L of the iris assembly 380. This position also corresponds to the protrusion(s) 388 of each contact member 382 being at their farthest position along radial slots 392 away from the longitudinal axis L of the iris assembly 380, and the protrusion(s) 388 being at their position along spiral-shaped grooves 372 as close to the first terminal end of the spiral-shaped groove 372 as the configuration allows (e.g. it should be understood that the protrusion 388 of each contact member 382 will be at a different position relative to the first terminal end of the corresponding spiral-shaped groove 372 in this initial position). With the crimper 300 in this initial state with the iris assembly 380 fully (or nearly fully) open, a user may place the device to be crimped within the interior of the opening of the iris assembly 380. In some examples, the device is a balloon-expandable prosthetic heart valve that is in an expanded condition and which is positioned over an inflated balloon of a delivery device.

When the user is ready to start crimping the device within the open iris assembly 380, the user need only rotate the handle 322 in the correct direction. Rotating the handle 322 causes the pinion 342 to rotate in the same direction, which forces the large gear 362 to rotate in the opposite direction. Rotation of the large gear 362 forces the cam track 370 to rotate in unison. As the cam track 370 rotates, the contact members 382 remain rotationally stationary at least due to the protrusions 388 being confined within respective radial slots 392 of the first crimping guide 390, the first crimping guide 390 being fixed to the housing assembly 310. Thus, as the cam track 370 rotates, the spiral grooves 372 rotate, forcing the protrusions 388 within the spiral grooves 372 to move radially inwardly, with that radial inward movement being guided and limited by the corresponding radial slot 392 which remains stationary. The rotation of the cam track 370 thus causes all of the contact members 382 to move radially inwardly in unison to reduce size of the opening of the iris assembly 380, with the assembly of contact arms 386 maintaining a generally circular or cylindrical shape that reduces in diameter as the cam track 370 rotates. The contact arms 386 press on the device within the opening of the iris assembly 380 as the contact arms 386 move radially inwardly, applying force on the device that causes the device to collapse or crimp. In the case of a balloon-expandable prosthetic heart valve, as the contact arms 386 are forced radially inwardly, the frame of the prosthetic heart valve will begin to collapse, as will the balloon positioned within the prosthetic heart valve as inflation media (e.g. saline) is forced out of the balloon due to the crimping forces. The user may continue rotating the handle 322 to further crimp the device until the device reaches a desired minimum diameter.

In some examples, the user may rotate the handle 322 until, based on visual or other observation, the device within the iris assembly 380 has reached the desired diameter. However, in other examples, one or more mechanical stops may be provided in order to stop the crimping process at a certain point, for example at a set desired minimum diameter of the opening of the iris assembly 380, which may help prevent over-crimping which could damage the device (e.g. a prosthetic heart valve) within the crimper 300, or even damage components of the crimper 300 itself.

Crimper 300 is merely one example of a manual crimper. Another example of a crimper which includes a single lever for crimping is described in more detail in U.S. Pat. No. 7,530,253, the disclosure of which is hereby incorporated by reference herein. The current state of the art of implantations of balloon-expandable prosthetic heart valves generally entails a human, who is on-site, manually operating a manual crimper (such as crimper 300 or any other prior art manual crimper) to collapse the prosthetic heart valve onto the delivery device just prior to implantation. This crimping may occur in the procedure room or in a nearby room. Often, the person operating the manual crimper on-site is an employee or other agent of the manufacturer of the prosthetic heart valve. This is, at least in part, because a significant amount of variation may occur during the crimping process when the crimping is performed manually. This variation may exist despite various features used in the manual crimper to try to reduce procedure-to-procedure crimping variation. Thus, employing designated personnel affiliated with the prosthetic heart valve manufacturer who are experts in crimping the prosthetic heart valve may be necessary, or at least desired, to provide more consistent results of the prosthetic heart valve being crimped onto the delivery device.

As described above, a balloon inflation system 170 may be implemented in an implantation of a prosthetic heart valve such as prosthetic heart valve 10 to provide one or more benefits, such as a more streamlined implantation procedure. Because the balloon inflation system 170 may already include or be operably connected to power (e.g. connection to a battery and/or AC mains) and may include or be operably connected to “smart” components such as processor, memory, sensors, etc. to help control inflation of the balloon 136, in some embodiments, it may be desirable to harness the balloon inflation system 170 to automate, or semi-automate, the process of crimping the prosthetic heart valve. Using balloon inflation system 170, or a similar system, in conjunction with a crimper to automate (or semi-automate) the crimping process my provide one or more benefits. In some examples, one or more of those benefits may be achieved without needing additional system components (or without needing many more system components), particularly if the balloon inflation system 170 would be used as part of the implantation procedure anyway.

One possible benefit of the use of balloon inflation system 170 (or a similar system) to automate or semi-automate crimping is improved crimping precision. As described in greater detail below, one or more sensors in (or operably coupled to) to the balloon inflation system 170 may measure force and/torque produced by an electromechanical actuator used to crimp the medical device (e.g. the prosthetic heart valve 10) to the desired diameter. One or more of these sensors, which may interface through a subsystem that includes an electrical sensor interface and a software algorithm that provide closed-loop control of the actuating mechanism, may help to achieve a precise target crimp diameter, and/or inward radial force on exerted on the medical device. This is in contrast to crimpers that are fully manually operated. For example, as noted above, even though manual crimpers may provide features to try to assist with achieving consistent crimping, such as employing mechanical hard stops to prevent over- or under-crimping, such manual crimpers are still sensitive to operator technique. Under-crimping, in the example of prosthetic heart valves, may create risk of patient vascular while delivering the prosthetic heart valve through the patient's vascular. Over-crimping, in the example of prosthetic heart valves, may present risk of damage to that prosthetic heart valve, which may render it less durable than one that was optimally crimped. The manual crimper example may rely on subjective measures to gauge whether a crimp is “good” or “bad”. On the other hand, an automated or semi-automated crimper may partially or entirely remove subject measures, while making it more likely that an optimal crimp profile may be achieved. Furthermore, because skill of the user may become less relevant or irrelevant, the use of designated personnel (e.g. employed by the prosthetic heart valve manufacturer) may be avoided, leading to further efficiencies and cost reductions.

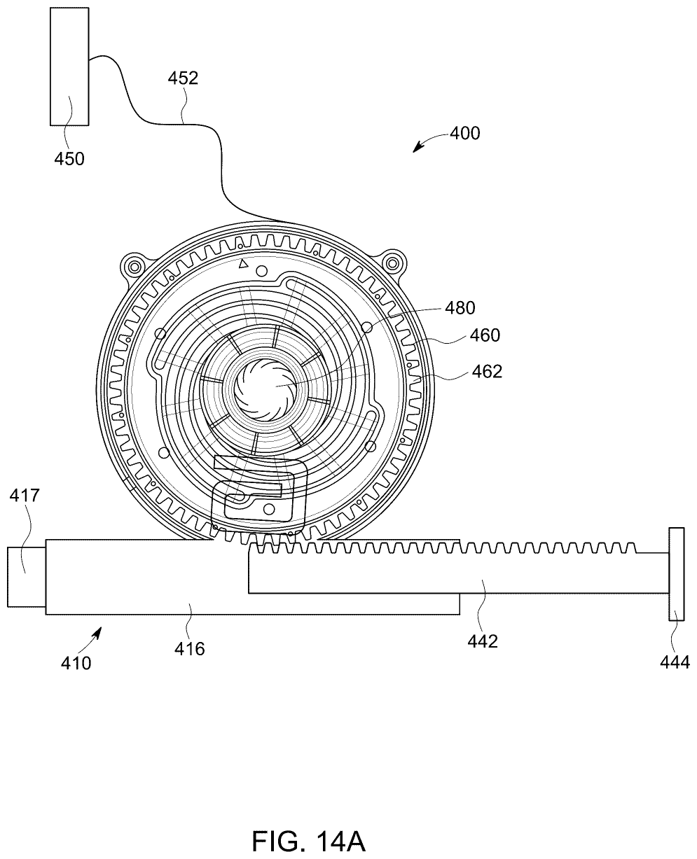

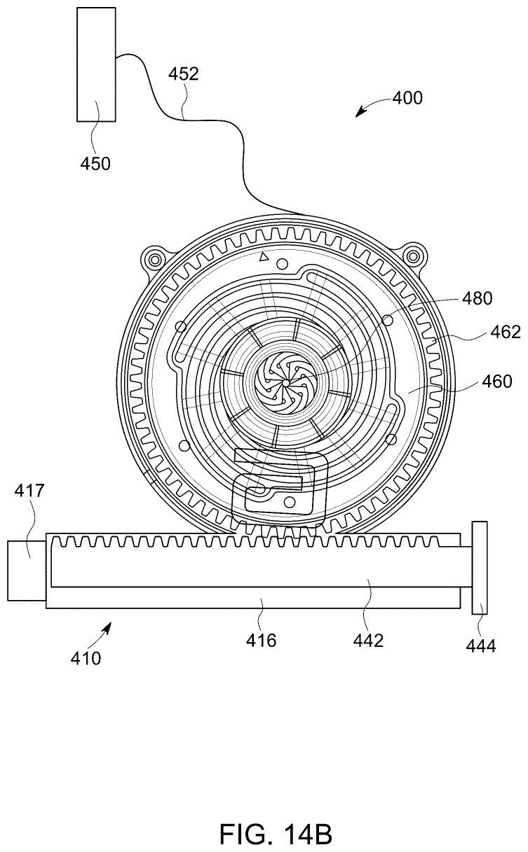

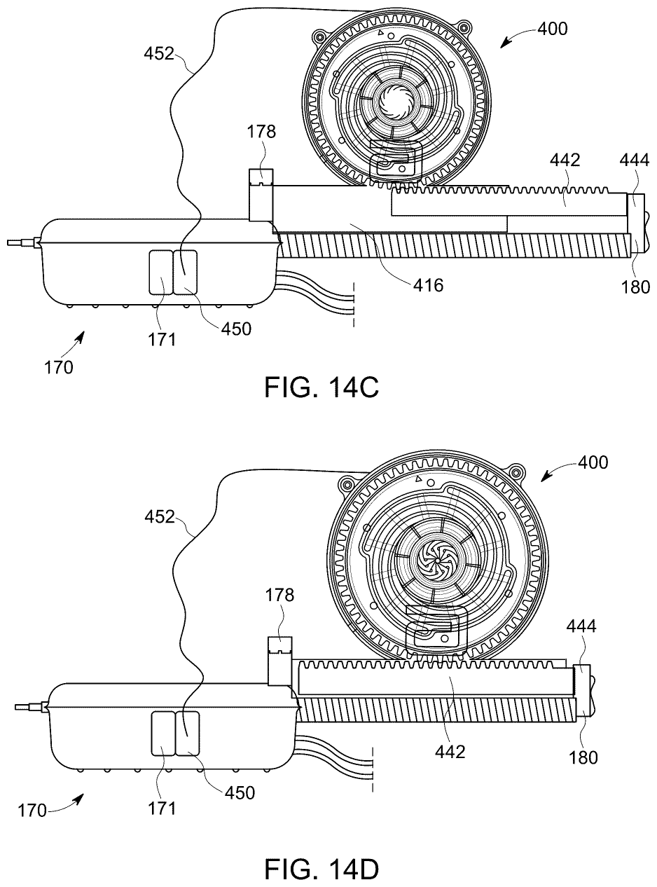

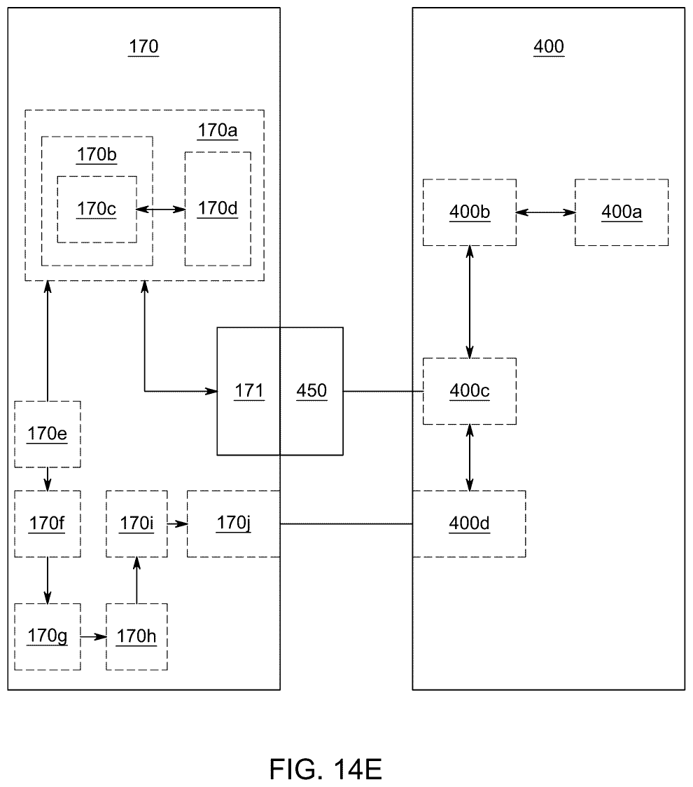

Referring now to FIG. 14A, one example of a crimper 400 is shown that may be configured for automated or semi-automated use, for example with a balloon inflation system (which may be generally similar or identical to balloon inflation system 170). FIG. 14A shows the crimper 400 with the iris assembly 480 in an open condition. The crimper 400 may have a number of components that are similar or identical to crimper 300. For example, crimper 400 may include a large gear assembly 460, including a large gear 462, that drives an iris assembly 480 to open or close, in substantially the same or identical fashion as described in connection with crimper 300. Thus, components such as crimping members, a first crimping guide with radial slots, and a second crimping guide with spiral grooves that acts as a cam track, are not described again in connection with crimper 400.

One main difference between crimper 300 and crimper 400 is that crimper 400 includes a linearly-activated driving member to cause rotation of the large gear 462, and thus a change in the size of the iris assembly 480. In the particular embodiment illustrated in FIG. 14A, the linearly-activated driving member may be a linear gear, for example a toothed rack 442, which may intermesh with gear teeth of large gear 462 such that linear motion of rack 442 drives rotation of large gear 462 and thus opening or closing of iris assembly 480. The rack 442 in some examples includes a proximal end that has a handle 444 that may be shaped similar to plunger handle 182 of syringe 174. The rack 442 may extend through an open end of the base 416 of base assembly 410, and may be configured to be driven into or pulled out of the base 416. In some examples, guide members such as bearings, wheels, rails, etc. may be provided within base 416 to help stabilize the position of the rack 442 as it is driven into or out of the base 416.

Another difference between crimper 300 and crimper 400, in addition to the swapping of a rotational pinion gear 342 with a linear toothed gear rack 442, is the inclusion of a connector plug 450, which may be a pigtail connector, with plug 450 being coupled to the crimper 400 via a wired connection 452. As is explained in greater detail below, the crimper 400 may include one or more sensors therein that may provide information through the connector plug 450 for use during crimping, although in other embodiments, such sensors may be omitted from the crimper 400, in which case connector plug 450 and wired connection 452 may also be omitted.

FIG. 14B shows the crimper 400 after the rack 442 has been fully driven into the base 416 of housing assembly 410, for example by pushing rack handle 444 toward the base 416. FIG. 14B shows that, with the rack 442 driven into the base 416, the teeth of the rack 442 have caused the large gear 462 to rotate via meshing of the gear teeth, and eventually closing of the iris assembly 480. It should be understood that rotation of the large gear 462 causes rotation of the cam track with spiral grooves, but for simplicity of illustration, the position of the cam track with spiral grooves is the same in FIGS. 14A-B. Further, the directionality of the spiral grooves may be opposite than they are shown in FIGS. 14A-14D to achieve closing of the iris assembly 480 by advancing rack 442 into the base 416. The directionality of the spiral may be selected to allow for closing of the iris assembly 480 when the rack 442 is either advanced into the housing 416, or retracted from the housing 416, depending on the particular desired configuration.

FIG. 14C shows one example configuration in which crimper 400 is mounted or otherwise coupled to balloon inflation system 170, although it should be understood that crimper 400 may be mounted or coupled to other examples of a balloon inflation system similar to balloon inflation system 170. It should also be understood that the crimper 400 may be mounted to a system that functions similarly to balloon inflation system 170, even if that system is not intended for use in actually inflating a balloon of a delivery catheter. While FIG. 14C shows the crimper 400 with the iris assembly 480 in a relatively open condition, FIG. 14D shows the crimper 400 with the iris assembly 480 in a relatively closed condition. It should be understood that the example of FIGS. 14C-D, one particular mechanical coupling mechanism is shown between the crimper 400 and the balloon inflation system 170, but various other types of mechanical couplings that allow for the function described below may be suitable. In the illustrated example, the handle 444 of the rack 442 may be received within a cradle of the moving member 180, similar to how plunger handle 182 is received. An end of the base 416 (opposite the open end through which rack 442 is received), may engage the housing 172 of the balloon inflation system 170 in any desired fashion to help stabilize the crimper 400 during crimping. For example, the distal end of the base 416 may include a projection 417 with flanges, which may be shaped similar to the proximal end of the outer body of the syringe 174. With this configuration, the proximal cradle 178 of the balloon inflation system 170 may be snapped over the distal projection 417 of the base 416 in order to lock the crimper 400 (other than the rack 442 and handle 442) to the housing 172 of the balloon inflation system 170. As noted above, however, various other options may be used to fix the main portion of the crimper 400 to the housing 172 of the balloon inflation system 170, and the rack 442 to the moving member 180.

In some embodiments, including those shown in FIGS. 14C-D, the crimper 400 may be electrically coupled to the balloon inflation system 170 in addition to being physically coupled. For example, the connector plug 450 of the crimper 400 may be connected to a corresponding receiving plug 171 of the balloon inflation system 170. The position of the receiving plug 171 is shown on a side of the housing 172, but it should be understood that the receiving plug 171 may be positioned differently, and in some cases, may be a receiving plug that is already provided with the balloon inflation system 170 for coupling other devices, such as delivery catheter 130, so that only one receiving plug is needed despite different components being able to electrically couple to the balloon inflation system 170.

In one exemplary use that involves prosthetic heart valve 10, the crimper 400 may be mechanically coupled to the balloon inflation system 170 so that the base 416 of the crimper 400 is fixed to the housing 172 of the balloon inflation system 170, and the rack 442 (e.g. via handle 444) may be coupled to the moving member 180 of the balloon inflation system 170. Preferably, this mechanical coupling is performed with the iris assembly 480 of the crimper 400 in the open condition. Prior or after the mechanical coupling of the crimper 400 to the balloon inflation system 170, the crimper 400 may be electrically coupled to the balloon inflation system 170, for example by connecting plug 450 to receiver 171. However, in some examples, the electrical connection step may be entirely omitted.

With the crimper 400 mechanically (and optionally electrically) coupled to the balloon inflation system 170, the user may position the medical device to be crimped within the open iris assembly 480. For example, prosthetic heart valve 10 may be in an expanded condition and positioned over balloon 136 while the balloon 136 is inflated. With the prosthetic heart valve 10 within the opening of the iris assembly 480, the user may operate the balloon inflation system 170 to drive the moving member 180 toward the housing 172 of the balloon inflation system 170. Movement of the moving member 180 forces the rack 442 to advance into the base 416, with the moving rack 442 causing rotation of the large gear 462, and thus closing of the iris assembly 480 to force the prosthetic heart valve 10 to collapse over the balloon 136 (and the balloon 136 may also begin to collapse for example by inflation media being forced out of the balloon 136). As noted above, the directionality of the spirals may be reversed compared to that shown in order for advancement of the rack 442 into the housing 416 to cause the iris assembly 480 to close, as opposed to open. Otherwise, if the spirals have the configuration shown in FIGS. 14C-D, the moving member 180 may withdraw the rack 442 from the base 416 to force the iris assembly 480 to close. Either way, the process may continue until the iris assembly 480 has reached its minimum desired diameter corresponding to a minimum desired diameter of the prosthetic heart valve 10.

FIG. 14E is a schematic view of crimper 400 coupled to balloon inflation system 170. It should be understood that additional components not shown in FIG. 14E may be included in one or both of crimper and balloon inflation system 170, and components shown in FIG. 14E may be omitted as desired.

On the right of FIG. 14E, crimper 400 is shown, which may include a housing 400a (which may include, for example, base 416 and any shells or other housing components). In addition to the housing 400a, the crimper 400 may include an iris cam driver 400b. The iris cam driver 400 may be, for example, one of (or a combination of) the rack 442, handle 444, large gear assembly 460 and/or iris assembly 480. The crimper 400 may also include one or more sensors 400c. Any sensors that can provide useful information may be used with crimper 400. In one example, sensor 400c includes a force sensor, which may measure any relevant force. A force sensor 400c may for example measure the force being applied onto the device (e.g. prosthetic heart valve) within the crimper while the iris assembly 480 is closing. In another example, sensor 400c includes a position sensor, which may measure the position of any component of the crimper 400 that can correlate to the size of the iris assembly 480, including for example the radial position of the crimping members, the rotational position of the large gear 462, and/or the linear position of the rack 442. In some examples, both force and position sensors may be provided on the crimper 400. As explained in greater detail below, in some examples, sensor(s) 400c may be omitted from crimper 400, and instead a force and/or position sensor on balloon inflation system 170 may be used to receive feedback about crimping status, in which case the electrical connection between the crimper 400 and balloon inflation system 170 may be omitted entirely. Still referring to the particular embodiment of FIG. 14E, the crimper 400 is shown with a mechanical interface 400d to mechanically coupled to the balloon inflation system 170. For example, the mechanical interface 400d may be one or both of the handle 444 and/or projection 417, although as explained elsewhere, different mechanical couplings may be used instead of the particular one(s) shown. Still further, crimper 400 is shown in FIG. 14E with a connector, which may be plug 450. Although not specifically represented in FIG. 14E, the connector or plug 450 may additionally contain signal wires to identify the particular model number or variant of the crimper 400, and may additionally be used to send a unique identifier that the firmware 170b of the balloon inflation system 170 reads to securely authenticate the crimper 400 as to prevent non-approved and/or non-compatible crimpers from being used with the balloon inflation system 170.

On the left of FIG. 14E, balloon inflation system 170 (which may also be referred to herein as a smart inflation device or “SID”) is shown, which may include a connector or receiving plug 171, shown in FIG. 14E as coupled to plug 450. Other exemplary components of balloon inflation system 170 are shown in FIG. 14E, including one or more microprocessors 170a, which may for example run firmware 170b including a control algorithm 170c, which may be operatively coupled to one or more memory storage devices 170d. In one example, the balloon inflation system 170 may include one or more batteries 170e, including for example one battery to supply power to the microprocessor 170a (and/or other internal circuitry) and another large rechargeable battery pack (which may be removable) to supply power to the motor 170g and/or other components such as sensors or other internal circuitry. In other examples, the battery 170e used to power the motor 170g may be replaced with an AC mains or other connection, which may be provided instead of or in addition to a battery 170e to power the motor 170g. Balloon inflation system 170 may also include motor driver circuitry 170f which may be operably coupled to a motor 170g, which may be for example a stepper motor. The motor 170g may be operably coupled to a drive mechanism, such as a leadscrew 170h. As described above in connection with balloon inflation system 170, the motor 170g may rotate the leadscrew 170h to drive the leadscrew 170h linearly into or out of the housing of the balloon inflation system 170. The leadscrew 170h may be coupled to an arm 170i, which may be, for example, the moving member 180 that receives the handle 444 (or another component) coupled to rack 442. The balloon inflation system 170 is also shown in FIG. 14E with a mechanical interface 170j mechanically coupled to the mechanical interface 400d of the crimper 400. Mechanical interface 170j may take any suitable form, including for example one or both of moving member 180 and/or proximal cradle 178.

As should be understood from the above description, the movement and forces supplied by the balloon inflation system 170 are capable of being used to actuate a syringe (such as syringe 174) to control inflation of a balloon of a delivery system (such as balloon 136 of delivery system 100) to deploy a prosthetic heart valve (such as prosthetic heart valve 10), while also being capable of actuating the iris assembly 480 of crimper 400 to crimp a prosthetic heart valve (such as prosthetic heart valve 10) onto a balloon of a delivery system (such as balloon 136 of delivery system 100). In some examples, the balloon inflation system 170 and/or crimper 400 may include a sterile cover, drape, or shell that allows one or both components to be re-used for more than a single case. Some examples of such drapes and their use with balloon inflation systems (which may be suitable for use instead of the particular embodiment shown herein) are described in greater detail in U.S. Patent Application Publication No. 2025/0205048, the disclosure of which is hereby incorporated by reference herein.

In the exemplary use of crimper 400 described in connection with FIGS. 14A-14D above, it is described that, once the crimper 400 is mechanically secured and electrically connected to the balloon inflation system 170, the prosthetic heart valve 10 and balloon 136 are co-axially placed inside the iris assembly 480. In some examples, alignment aids, such as fiduciaries or mechanical hard-stops may aid the user in optimal positioning of the prosthetic heart valve 10 within the iris assembly 480. When the operator is ready to crimp, a button may be depressed or otherwise actuated, which button may be on the crimper 400, the balloon inflation system 170, and/or a tablet or other input screen operably coupled with the balloon inflation system 170 (and/or crimper 400) to initiate motion of the arm 170i. Motion and forces of the arm 170i may be translated, via mechanical interfaces 170j, 400d to the rack 442 (and/or handle 444) or other iris cam driver 400b relative to the crimper housing 400a (e.g. crimper base 416). This motion may in turn rotates a cam mechanism, such as large gear assembly 460, that drives the iris assembly 480 to reduce in inner diameter and crimp the prosthetic heart valve 10 onto the balloon 136.

As crimping is performed on the prosthetic heart valve, the one or more sensor(s) 400c may provide feedback to the balloon inflation system 170 regarding the position of any moving component of the crimper 400 and/or forces being applied to the prosthetic heart valve (and/or resistance forces being applied by the prosthetic heart valve back to the crimper 400). The balloon inflation system 170 may receive the information from the one or more sensor(s) 400c to determine whether the iris assembly 480 has reduced to the desired size and/or whether the maximum desired crimping force(s) have been applied to the prosthetic heart valve to achieve a minimum desired diameter. Once the feedback from the sensor(s) 400c indicates that the desired minimum size of the prosthetic heart valve 10 has been reached, the microprocessor 170a and/or motor driver circuitry 170f may cause the motor 170g to stop causing crimping, or even to reverse direction to cause the iris assembly 480 to begin to open. For example, the rotational position of the large gear 462 (and/or the linear position of the rack 442) may have a one-to-one correlation with the interior size of the iris assembly 480, and thus to the crimped diameter of the prosthetic heart valve 10, and one or more of these positions may be monitored by sensor(s) 400c to determine the progress of the crimping process. It should be understood that various types of controls may be implemented using the microprocessor 170a and/or motor driver circuitry 170f, for example in which the motor 170f applies substantially even crimping speed (and/or force) during the entire crimping process, or in which the crimping proceeds at a first relatively fast pace for an early part of the crimping but proceeds at a second relatively slow pace as the crimping process nears the end in which the prosthetic heart valve 10 has reached its minimum desired size. It should also be understood that the particular minimum desired size of the prosthetic heart valve 10 may be selected by the user prior to crimping, for example by inputting a desired minimum size of the prosthetic heart valve 10 and/or iris assembly 480 into the balloon inflation system 170 (e.g. via a table or other input device operably coupled to the balloon inflation system 170). In some examples, the minimum desired size of the prosthetic heart valve 10 may depend on the particular model and/or size of the prosthetic heart valve 10, and a number of pre-programmed size options may be provided that correspond to the one or more options for the particular model and/or size of the prosthetic heart valve 10, such that the user only needs to select the model and/or size of the prosthetic heart valve 10 to set a desired minimum crimp size, as opposed to entering an actual size. In still other examples, the crimper 400 may be tailored for use with the specific prosthetic heart valve 10 (or with a sub-group of prosthetic heart valves that all have the same minimum crimping size) such that, when the crimper 400 is electrically coupled to the balloon inflation system 170 (e.g. via plug 450 and plug receiver 171), a unique identifier is provided by the crimper 400 to the balloon inflation system 170 which is recognized by the microprocessor 170a and automatically sets the desired minimum crimping size of the prosthetic heart valve 10.

Although the electrical interface between the crimper 400 and the balloon inflation system 170 is shown at separate locations from the mechanical interfaces between the two components, in other examples, the electrical connectors may be provided at the mechanical interfaces. For example, electrical connectors may be provided on the proximal cradle 178 and the projection 417, or on the moving member 180 and the handle 444, or on any other two components the mechanically connect the balloon inflation system 170 to the crimper 400 so that the step of mechanically coupling the balloon inflation system 170 and the crimper 400 may be performed simultaneously with electrically coupling the balloon inflation system 170 and the crimper 400.

As noted above, although one or more sensors 400c may be provided with crimper 400 to assist in controlling the progress of the crimping, in other embodiments, one or more sensors may be provided with the balloon inflation system 170. For example, the motor current of the motor 170g of the balloon inflation system 170 may be monitored, forces on one or more load cells on the arm 170i may be monitored, and/or one or more position sensors on the arm 170i may be monitored, to determine how progress of the crimping should continue (including determining when to stop the crimping). If one or more sensors of the balloon inflation system 170 are used to monitor the progress (and thus control the progress) of the crimping process, the sensor(s) 400c of the crimper 400, and/or the electrical connection between the crimper 400 and the balloon inflation system 170, may be omitted.

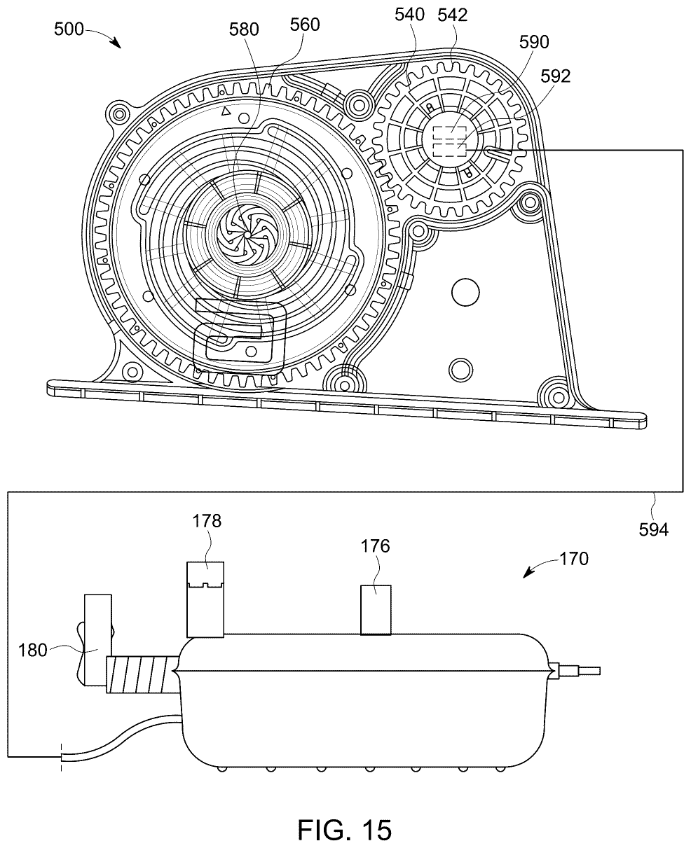

FIG. 15 illustrates another example of a crimper 500 used with a balloon inflation system 170 that is highly similar to that shown and described in connection with FIGS. 14A-14E, with two main differences. The first main difference is that the balloon inflation system 170 is only electrically, as opposed to mechanically, coupled to the crimper 500. The second main difference is that, due to the lack of mechanical connection, the crimper 500 includes its own motor 590 that is provided power via the electrical connection 594 to the balloon inflation system 170. Crimper 500 may be substantially similar or identical to crimper 300, except that instead of a handle assembly 320 manually driving a pinion gear assembly 340, the motor 590 drives the pinion gear assembly 540. Thus, the iris assembly 580 may be similar or identical to iris assembly 380, and the large gear assembly 560 may be similar or identical to the large gear assembly 360, and thus these components are not described in detail here again. The pinion gear 542 may be identical (or substantially similar) to the pinion gear 342, other than the fact that it is driven by a motor 590 internal to the crimper 500, instead of a handle 322 external to the crimper 300.

FIG. 15 shows a motor 590 internal to the pinion gear 542, although the precise position of the motor 590 may be different as long as operation of the motor causes operation of the pinion gear 542. The balloon inflation system 170 may include an electrical connection 594, which may be similar to plug 450, that electrically connects to a plug receiver 592 on the crimper 500. It should be understood that in other embodiments, the crimper 500 may include the plug that gets connected to a plug receiver of the balloon inflation system 170, or any other connectors may be suitable to electrically couple the balloon inflation system 170 to the crimper 500. The components of balloon inflation system 170 shown in FIG. 14E, with the exception of the mechanical interface 170j, may apply to the balloon inflation system 170 of FIG. 15. For example, the balloon inflation system 170 may include one or more batteries 170c or other power source such as a connector to electrical mains in order to transmit power to the motor 590 via the electrical connection 594 to plug receiver 592. Similarly, the components of crimper 400 shown in FIG. 14E may generally apply to crimper 500, except that the mechanical interface 400d may be omitted. All the other description regarding the control and use of the crimper 400 with balloon inflation system 170 may apply to the control and use of the crimper 500 with balloon inflation system 170, with the sole exception that the mechanical connections may be omitted, and the large gear assembly 560 is driven by a pinion gear assembly 540 with a motor 590 internal to the crimper 500. Thus, for example, the crimper 500 and/or the balloon inflation system 170 may include the sensors to determine and control the progress of the crimping, and the balloon inflation system 170 may include the control mechanisms (e.g. microprocessor 170a) to drive the motor 590. In some examples, the balloon inflation system 170 may include motor driver circuitry 170f that not only drives the motor 170g of the balloon inflation system 170, but also the motor 590 of the crimper 500. In other examples, the balloon inflation system 170 may include separate motor driver circuitry for driving the motor 590, or otherwise the crimper 500 may include motor driver circuitry for driving the motor 590. Other than these differences, the use of crimper 500 with the assistance of balloon inflation system 170 may be substantially identical to the use of crimper 400 with the assistance of balloon inflation system 170.

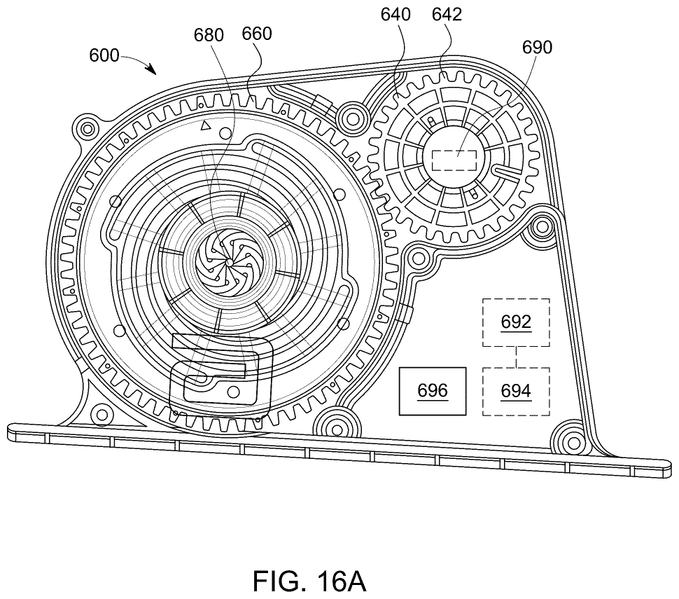

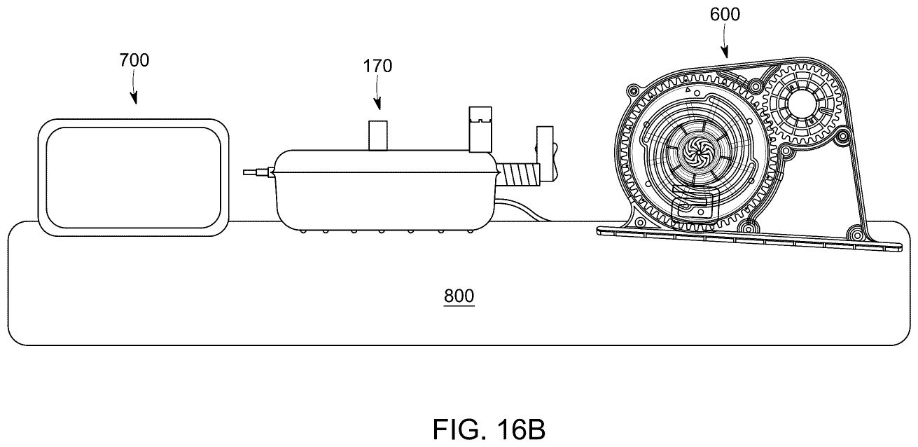

FIG. 16A illustrates another example of a crimper 600 that is highly similar to crimper 500 shown and described in connection with FIG. 15, with at least one main difference. Like crimper 500, crimper 600 may include an internal motor 690. However, unlike crimper 500, crimper 600 includes an internal power source, such as a battery 692, which may be a rechargeable battery, and/or connections to AC mains. Because crimper 600 includes its own motor 690 and its own power source 692, the crimper 600 is capable of functioning independently of the balloon inflation system 170. Crimper 600 may be otherwise substantially similar or identical to crimper 500. For example, the iris assembly 680 may be similar or identical to iris assembly 580, the large gear assembly 660 may be similar or identical to the large gear assembly 560, and the pinion 642 and the pinion gear assembly 640 may be similar or identical to the pinion 542 and the pinion gear assembly 540, and thus these components are not described in detail here again.

As shown in FIG. 16A, the crimper 600 may include its own power source 692, which is preferably a rechargeable battery, which is operably coupled to motor 690 such that the power source 692 can provide power to the motor 690. To allow the crimper 600 to function fully independently of the balloon inflation system 170, the crimper 600 may also include one or more microprocessors 694, which may include firmware, control algorithms, as well as one or more memory modules and/or additional circuitry such as motor driver circuitry, which may all be generally similar to that shown and described in connection with the balloon inflation system 170 of FIG. 14E. In some examples, crimper 600 may also include an interface 696, which may be dedicated physical buttons, a graphic user interface (“GUI”), or any other component that allows for input and/or is capable of displaying relevant information. For example, the interface 696 may include basic controls, such as a “start” and “stop” button to initiate or stop crimping, respectively. The interface 696 may also include indicators, such as LED backlit indicators of the charge level of the battery 692 and/or of the current operating status of the crimper 600. The interface 696 may include (or otherwise the crimper 600 may include) a wired connection port or a wireless communication feature (such as a Bluetooth and/or Near Field Communication (“NFC”) subsystem) that enables the crimper 600 to communicate with another device, such as a tablet 700 or another computing device. The tablet 700 or other computing device may provide a user interface (which may be in addition to or instead of the user interface 696 on the crimper 600 itself) to allow for control and status/operational indications of the crimper 600. It should be understood that tablet 700 (or another computing device) may be used similarly with the balloon inflation system 170 to provide controls for, and information relating to the use of, balloon inflation system 170. In other words, a single tablet 700 or other computing device may provide for control of, and display of information relating to, both the crimper 600 and the balloon inflation system 170 (if such a balloon inflation system 170 is used with the crimper 600, which as described above is not required). If the power source of the crimper 600 is a rechargeable battery 692, it may be recharged with its own AC mains charger or, in other embodiments, the charging interconnect may be implemented within a charging dock 800, shown in FIG. 16B. If the battery 692 is chargeable via a charging dock 800, the same charging dock 800 may also be configured to recharge the battery of the balloon inflation system 170 and/or the table 700 (or other computing device).

The use of crimper 600 may be largely similar to crimper 500, with the main difference being that the crimper 600 does not need to be electrically connected to the balloon inflation system 170 for providing power. Also, to the extent sensors are included, such as position and/or force sensors that allow for enhanced control of the crimper 600 (e.g., allowing the crimping to proceed following manual initiation until the crimper 600 automatically stops the crimping once the prosthetic heart valve 10 has reached the desired minimum diameter) are provided as part of the crimper 600, as opposed to part of the balloon inflation system 170 which is an option described in connection with crimper 500. All of the other controls described above, including for example the ability to automatically slow the speed of the crimper 600 as the prosthetic heart valve nears its final desired minimum diameter, may apply with substantially equal force to crimper 600.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A system for crimping and expanding a medical device, the system comprising:
a delivery system having a handle, a catheter extending from the handle, and an inflatable balloon near a distal end of the catheter;
a balloon inflation system;
a syringe configured to receive inflation media therein, the syringe configured to be received on the balloon inflation system and to be coupled to the delivery system; and
a crimper, the crimper having an iris assembly configured to receive the medical device therein, the iris assembly being configured to transition from a first larger diameter to a second smaller diameter, the crimper being configured to mechanically couple to the balloon inflation system;
wherein when the crimper is mechanically coupled to the balloon inflation system, the balloon inflation system is configured to actuate the crimper to transition the iris assembly from the first larger diameter to the second smaller diameter to crimp the medical device onto the balloon when the medical device is received within the iris assembly;
wherein when the syringe (i) has inflation media received therein, (ii) is received on the balloon inflation system, and (iii) is coupled to the delivery system, the balloon inflation system is configured to actuate the syringe to advance the inflation media from the syringe to the balloon to expand the balloon and to expand the medical device that is crimped onto the balloon.

2. The system of claim 1, wherein the balloon inflation system includes a moving member and a cradle, the moving member being translatable toward or away from the cradle.

3. The system of claim 2, wherein the syringe has a barrel configured to be received within the cradle, and a plunger handle configured to couple to the moving member.

4. The system of claim 2, wherein the crimper includes a spur gear coupled to a cam track so that rotation of the spur gear causes rotation of the cam track, and rotation of the cam track transitions the iris assembly from the first larger diameter to the second smaller diameter.

5. The system of claim 4, wherein the crimper includes a rack gear, the rack gear being translatable into or out of a base of the crimper, translation of the rack gear causing rotation of the spur gear.

6. The system of claim 5, wherein the rack gear includes a rack gear handle, the rack gear handle configured to be coupled to the moving member of the balloon inflation system.

7. The system of claim 6, wherein the crimper includes a distal projection, the distal projection configured to be received within the cradle of the balloon inflation system.

8. The system of claim 1, wherein the balloon inflation system includes a motor.

9. The system of claim 8, wherein the crimper does not include a motor.

10. The system of claim 1, wherein the crimper is configured to be electrically connected to the balloon inflation system.

11. The system of claim 1, wherein the crimper includes at least one sensor.

12. The system of claim 11, wherein the at least one sensor is a position sensor.

13. The system of claim 11, wherein the at least one sensor is a force sensor.

14. The system of claim 1, further comprising the medical device.

15. The system of claim 14, wherein the medical device is a balloon-expandable prosthetic heart valve.

16. A method comprising:
positioning a medical device on an inflatable balloon that is coupled to a distal end of a catheter of a delivery system while the medical device is in an expanded condition;
while the medical device is in the expanded condition on the balloon, positioning the balloon and the medical device within an iris assembly of a crimper while the iris assembly has a first large diameter;
mechanically coupling the crimper to a balloon inflation system, either before or after positioning the balloon and the medical device within the iris assembly of the crimper;
actuating the balloon inflation system a first time, while the crimper is mechanically coupled to the balloon inflation system, to transition the iris assembly from the first large diameter to a second smaller diameter to crimp the medial device into a collapsed condition on the balloon.

17. The method of claim 16, further comprising mechanically uncoupling the crimper from the balloon inflation system after the medical device is crimped on the balloon, and then mechanically coupling a syringe to the balloon inflation system, the syringe having inflation media therein.

18. The method of claim 17, wherein the medical device is a balloon-expandable prosthetic heart valve, and the method further includes advancing the crimped prosthetic heart valve into a patient until the crimped prosthetic heart valve is positioned within a native valve annulus of a patient.

19. The method of claim 18, further comprising actuating the balloon inflation system a second time, while the crimped prosthetic heart valve is positioned within the native valve annulus of a patient, to advance inflation media from the syringe, through the delivery system, and into the balloon to inflate the balloon and to expand the prosthetic heart valve into the native valve annulus of the patient.

20. The method of claim 16, wherein actuating the balloon inflation system the first time includes activating a motor within the balloon inflation system to translate a moving member of the balloon inflation system, whereby the moving member advances a rack gear of the crimper to rotate a spur gear of the crimper, rotation of the spur gear rotating a cam track such that rotation of the cam track transitions the iris assembly from the first large diameter to the second smaller diameter.
